(19) United States
(12) Patent Application Publication
(10) Pub. No.: US 2014/0303476 A1
(43) Pub. Date: Oct. 9, 2014

(54) Photoacoustic Imaging Using A Versatile Acoustic Lens

(71) Applicants: Vikram S. Dogra, Pittsford, NY (US); Navalgund A. H. K. Rao, Rochester, NY (US); Wayne H. Knox, Pittsford, NY (US)

(72) Inventors: Vikram S. Dogra, Pittsford, NY (US); Navalgund A. H. K. Rao, Rochester, NY (US); Wayne H. Knox, Pittsford, NY (US)

(73) Assignee: University of Rochester, Rochester, NY (US)

(21) Appl. No.: 14/308,325

(22) Filed: Jun. 18, 2014

Related U.S. Application Data
(63) Continuation of application No. 12/579,741, filed on Oct. 15, 2009, now abandoned.
(60) Provisional application No. 61/105,590, filed on Oct. 15, 2008.

(51) Int. Cl. A61B 5/00

(52) U.S. Cl. CPC A61B 5/0095; A61B 5/4887
USPC 600/407

(57) ABSTRACT

To image various soft tissues in the body using pulsed laser optical excitation delivered through a multi-mode optical fiber to create photoacoustic impulses, and then image the generated photoacoustic impulses with an acoustic detector array, a probe includes either a mirror and an acoustic lens or a special acoustic lens of variable focal length and magnification that can operate in a liquid environment that is aberration-corrected to a sufficient degree that high resolution images can be obtained with lateral as well as depth resolution.

### REFERENCE TO RELATED APPLICATIONS

The present application claims the benefit of U.S. Provisional Patent Application No. 61/105,590 (Confirmation No. 6495), filed Oct. 15, 2008. The invention disclosed in the present application is related to the invention disclosed in U.S. patent application Ser. No. 12/505,264 (Confirmation No. 1769), filed Jul. 17, 2009. The disclosures of both of those applications are hereby incorporated by reference in their entireties into the present disclosure.

### FIELD OF THE INVENTION

The present invention is directed to photoacoustic imaging and more particularly to such imaging using a multi-element acoustic lens.

### DESCRIPTION OF RELATED ART

Prostate cancer is the most prevalent newly diagnosed malignancy in men, second only to lung cancer in causing cancer-related deaths. Adenocarcinoma of the prostate is the most common malignancy in the Western world. There were a projected 218,890 new cases of prostate cancer diagnosed in the United States in 2007, with an estimated 27,050 deaths. As men age, the risk of developing prostate cancer increases. Prostate cancer has been found incidentally in approximately 30% of autopsy specimens of men in their sixth decade. Seventy to 80% of patients who have prostate cancer are older than 65 years. Clinically localized disease is usually suspected based on an elevated prostate specific antigen (PSA) test or abnormal digital rectal exam (DRE), prompting transrectal ultrasound (TRUS) guided biopsy of the prostate for definitive diagnosis. TRUS however, is not reliable enough to be used solely as a template for biopsy. There are cancers that are not visible (isoechoic) on TRUS. Furthermore, in PSA screened populations, the accuracy of TRUS was only 52% due to false-positive findings encountered. Increased tumor vessels (angiogenesis) have been shown microscopically in prostate cancer compared with benign prostate tissue. Efficacy of color and power Doppler ultrasound has not been demonstrated, probably due to limited resolution and small flow velocities. Elasticity imaging, with its many variants, is a new modality that is currently under extensive investigation. It is evident that given the limitations of the present diagnostic protocols, development of a new imaging modality that improves visualization and biopsy yield of prostate cancer would be beneficial. Furthermore, by making it cost effective, we can place it in the hands of primary care physicians, where it will serve its primary purpose as an adjunct to PSA, DRE, and TRUS.

The need for tumor visualization is equally critical in the treatment of localized prostate cancer disease. Existing therapeutic strategies, namely external beam radiation, prostate brachytherapy, cryosurgery, and watchful waiting, all will benefit significantly from the development of a new modality that promises better tumor contrast. Thus, prostate cancer continues to be an area in which progress is needed despite recent advancements.

Appropriate imaging of prostate cancer is a crucial component for diagnosing prostate cancer and its staging, in addition to PSA levels and DRE. The current state of prostate imaging for diagnosis of prostate cancer includes ultrasound, ultrasound-guided prostate biopsies, magnetic resonance imaging (MRI), and nuclear scintigraphy. These modalities are helpful, but have drawbacks and limitations. MRI is expensive and not mobile. Nuclear scintillation is expensive, provides low resolution planar images, and there are problems with radiotracer excretion through the kidneys. Both these modalities are not available for general use.

Ultrasound is not reliable enough to use solely as a template for diagnosing prostate cancer. It has two problems. First, in many cases prostate cancer appears as an isoechoic lesion (similar gray scale value as surrounding tissue) causing high miss rate. Secondly, when it is visible (hyper or hypoechoic), it is not possible to say with certainty if it is cancer or benign because many other noncancer conditions such as prostate atrophy, inflammation of the prostate gland, and benign tumors may also look similar in appearance on ultrasound examination. A biopsy has to be performed on the suspect lesion for definitive diagnosis. Biopsies are uncomfortable and bleeding may result as a complication. Because of poor lesion detection, even the current prostate biopsy techniques miss approximately 30% of prostate cancer. Utility of color flow and power Doppler in conjunction with gray scale ultrasound has been explored, but not successfully. Therefore, there is an urgent need for a new imaging methodology that will be portable, economical to build, and will have widespread utility as a tool for primary screening and diagnosis of prostate cancer.

### SUMMARY OF THE INVENTION

It is therefore an object of the invention to meet that need.

To achieve the above and other objects, the present invention is directed to an implementation of an acoustic lens/zoom acoustic lens or a combination of an acoustic lens and acoustic mirrors. The present invention addresses the need to improve signal to noise (S/N) ratio in medical photoacoustic imaging; however, a preferred embodiment will be targeted towards prostate gland imaging.

To image various soft tissues in the body using pulsed laser optical excitation delivered through a multi-mode optical fiber to create photoacoustic impulses, and then image the generated photoacoustic impulses with an acoustic detector array, at least some embodiments of the invention implement a special acoustic lens of variable focal length and magnification that can operate in a liquid environment that is aberration-corrected to a sufficient degree that high resolution images can be obtained with lateral as well as depth resolution.

### BRIEF DESCRIPTION OF THE DRAWINGS

Preferred embodiments of the present invention will be set forth below with reference to the drawings, in which:

FIG. 1A is a schematic diagram showing a probe for photoacoustic imaging of the prostate using an acoustic lens and mirror;

FIG. 1B is a schematic diagram showing a probe for photoacoustic imaging of the prostate using an acoustic lens without a mirror;

FIG. 2 shows a single biconcave acoustic focusing lens;

FIG. 3 shows a multi-element acoustic lens having positive and negative elements; and

FIG. 4 shows a multi-element acoustic lens with continuous variation of magnification.

### DETAILED DESCRIPTION OF THE PREFERRED EMBODIMENTS

Preferred embodiments of the present invention will be set forth in detail with reference to the drawings, in which like reference numerals refer to like elements throughout.

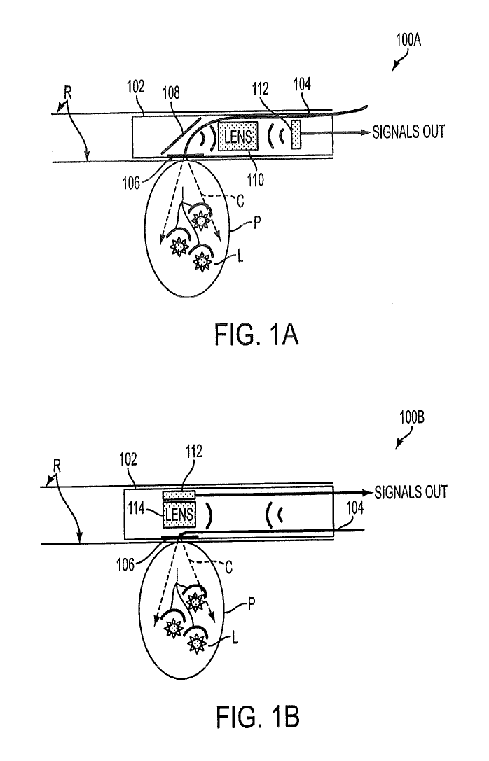

A first preferred embodiment provides prostate imaging through a rectal probe. FIG. 1A shows an example of imaging of the prostate with a probe 100A whose housing 102 is designed to be placed into the rectum. The probe 100A includes several elements. A multi-mode optical fiber 104 carries a laser pulse of certain energy in the range of ten nanoseconds duration in a wavelength range of 500-1500 nm wavelength. The fiber carries the laser energy to an acoustic and optic window 106, through which the laser energy passes to the rectal wall R, where it illuminates a portion of the prostate P. The fiber has a certain numerical aperture and illuminates the prostate with a cone of light C of certain angle. Typically, a fiber with NA=0.25 will illuminate within a 25 degree cone. The housing 102 would typically be sealed and filled with an appropriate liquid.

The laser wavelength is selected so as to be preferentially absorbed in lesions L which may contain an enhanced density of blood vessels. In such as case, light absorption is primary through hemo/deoxyhemoglobin, and wavelength in the range of 800 nm is preferred. The lesions of interest may also have enhanced infrared absorption by use of targeted probe molecules that attach only to the lesions or regions of interest and provide enhanced absorption of infrared radiation. The enhanced absorption in the lesions produces enhanced generation of photoacoustic impulses I that radiate out of the prostate in all directions. A certain fraction of such acoustic radiation penetrates the rectal wall R, passes through the acoustic and optic window 106, reflects off of a mirror 108 and is directed into a specially designed acoustic lens 110. The acoustic lens 110 then directly images the photoacoustic signals onto an image plane containing an acoustic detector array 112. The acoustic detector array 112 contains N×M elements (where N and M are selected during the design of the probe to give a required imaging resolution) that also provide time-resolved output so that the time domain information is available for depth-related image processing.

The acoustic mirror 108 shown in FIG. 1A could be made of certain metals such as copper or tungsten, or by a thin membrane such as Mylar that is mounted so as to include a thin air gap behind the membrane. This mirror could also be curved, in principle, so that it becomes part of the catadioptric imaging system.

FIG. 1B shows an alternate configuration 100B in which an acoustic mirror is not used. In this case, the optical axis of the lens 114 and detector imaging system 112 is perpendicular to the axis of the probe, requiring a more compact implementation of the lens 114. Both configurations include a window 106 which needs to be transparent to laser light and acoustic signals as well. This should be mechanically strong as well. A thin sapphire plate is an example of such a window material.

The design of the lens 110 or 114 will now be described.

Acoustic lenses function in some ways similarly to optical lenses. In optical systems, when the dimensions of the lenses, sources and image resolution elements are much greater than the optical wavelength, geometrical optics provides a good approximation for the purpose of lens and optical system design. In the case of acoustics, wavelengths of interest for the projects under consideration are in the range 0.2 to 5 mm. The acoustic energy can be described in a ray model, and rules similar to Snell's law of refraction apply to rays that are bent at interfaces between dissimilar materials. In the acoustic case, such ray bending is governed by the differences in the material properties such as the acoustic velocity, impedance, etc., which can be very different for various materials.

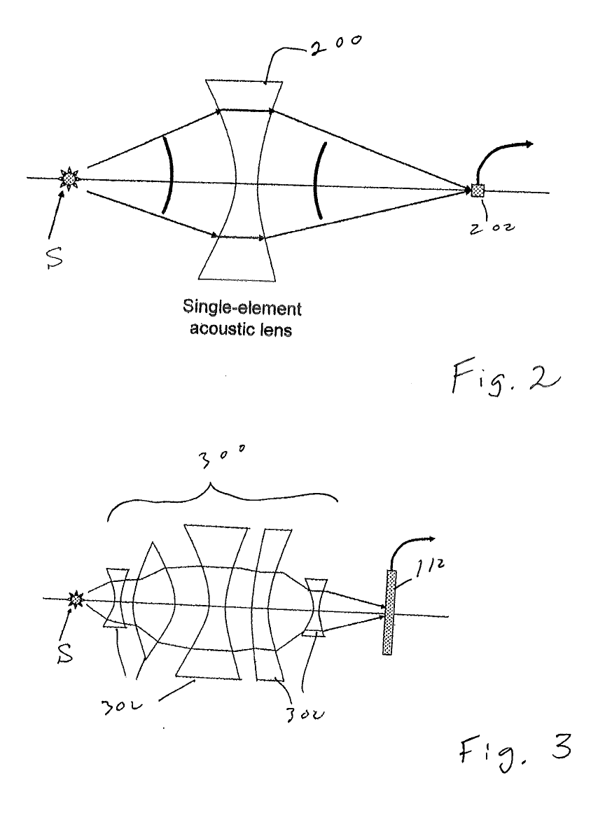

FIG. 2 shows a simple case of a single element 200. When the lens material has a higher sound velocity than that of the surrounding medium, a bi-concave lens provides a focusing action to focus acoustic waves from a source S onto a detector 202.

In the case of the present example of prostate lesion imaging through rectal access, the imaging conditions are severely constrained. The outside diameter of the probe must be no larger than 30 mm, and the total distance from the prostate wall to the detector array would be in the range 4-7 cm. A preferred embodiment of the invention would include a variable magnification “zoom lens” function so that wide angle scans could be first performed, and if smaller regions of interest are seen, higher magnification could be dialed in so as to provide enhanced levels of detail in those regions. Furthermore, it would be desirable to obtain acoustically diffraction-limited operation, in the sense that the acoustic lens is able to image the acoustic emissions of the small regions of interest at the highest resolution that is possible with perfect imaging, i.e., limited only by the diffraction effects of the radiation itself. This means that such an acoustic lens would have to be designed and constructed so as to provide diffraction-limited acoustic imaging.

All lens systems are subject to certain levels of aberrations such as spherical aberration, chromatic aberration, astigmatism, coma, and field curvature, which all need to be corrected in order to provide diffraction-limited imaging performance. Furthermore, the lens elements should exhibit high transmission in the wavelength range of interest and should be corrected for excessive reflections on the element surfaces. In the optical domain, high transparency is not difficult to achieve, and anti-reflection coatings can be applied to surfaces. In the acoustic domain, attention must be paid to the acoustic impedance matching of the interfaces in order to avoid excessive loss, and material losses are more problematic compared to the optical domain. It is desirable to provide new material options for design of high performance versatile acoustic lenses.

In order to simultaneously satisfy the requirements for aberration correction, intensity throughput, imaging quality and flexibility in performance, it is desirable to construct more complex acoustic lenses. FIG. 3 shows a schematic illustration of a multi-element lens 300. It includes various refractive devices 302, some with positive (focusing power) and some with negative (defocusing) power.

It is necessary to perform a complete acoustic design of such a complex lens system in order to optimize all the relevant aberrations and optimize performance. In the case of prostate imaging, the maximum lens aperture would be roughly 25 mm, and the total distance from source to detector would be in the range of 4-7 cm; therefore, the lens would be operating at nearly f/1 configuration. The range of capabilities is limited by the available acoustic materials. In the case of multi-element optical lens design, it is a standard technique to use a range of glasses that exhibit a range of dispersive and refractive features so as to optimize the lens system performance.

It is proposed to use hydrogel materials as acoustic lens elements. Such materials consist of a collection of different monomer materials that are mixed together in definite proportions and polymerized to create polymers that when immersed in water take up a predetermined proportion of water in the range of a few percent to as high as 80%. Correspondingly, the physical properties of these materials scale with the water proportion. A wide range of such hydrogels are available, including silicone-based materials and non-silicone-based materials. Silicone is widely used as a material for acoustic lenses, and silicone doped with nano-crystalline materials has been shown to exhibit low sound velocity and low acoustic attenuation. The important and relevant parameters for acoustic lens design are sound speed, acoustic impedance, attenuation, and figure of merit. The hydrogel material system is interesting for multi-element acoustic lens design because in one limit (near 0% water) such materials will exhibit acoustic properties similar to the familiar silicone materials, while in the opposite limit (80% water) hydrogels will exhibit acoustic properties closer to those of water. Therefore, we expect that there will be an almost linear scaling of all relevant acoustic material parameters in the range of available hydrogels and that these can be used to fabricate a range of elements for use in a multi-element acoustic lens such as shown in FIG. 3. It is necessary to measure relevant acoustic parameters of hydrogels of various formulations in order to determine the range of available options.

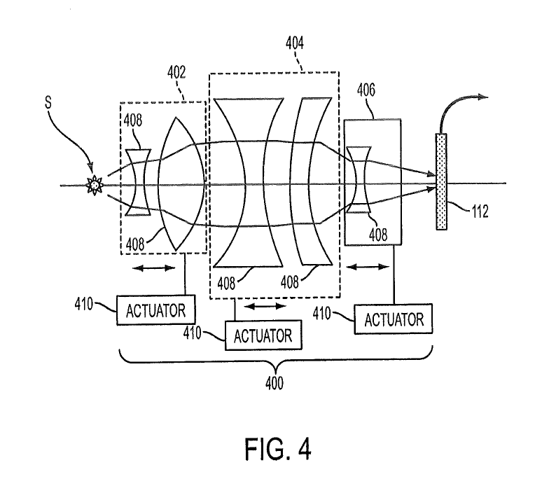

As mentioned previously, it is desirable to obtain performance in medical acoustic imaging that is equivalent to a “zoom lens” that is known in conventional photography. Such a lens can provide imaging over a continuously variable range of focal lengths or magnifications. This kind of functionality could be obtained by a specific design of a multi-element acoustic lens that incorporates movable elements, as is typically done with optical zoom lenses. FIG. 4 illustrates this concept. In the multi-element acoustic lens 400 of FIG. 4, several groups 402, 404, 406 of acoustic lens elements 408 are arranged to move in a prescribed motion under the control of actuators 410 so as to continuously vary the magnification of the image, while simultaneously maintaining optimized control of aberrations. In this kind of lens system, certain group of lenses such as group 402, group 404 and group 406 are arranged to provide motion in response to an external control such that the overall magnification changes continuously while maintaining optimized performance. This gives the system operator the ability to see gross features as well as the ability to “zoom in” to see greater detail.

The successful design of such a complex lens depends on the availability of adequate acoustic lens design software, as well as availability of detailed information on material properties-vs-relevant control parameter, which in the case of hydrogels would be the variation of key parameters-vs-water concentration. We note that in the design for the probe as shown in FIGS. 1A and 1B, is it likely that the probe 100A or 100B is completely sealed, and therefore the surrounding solution would be an additional degree of freedom that could include saline or oil or other content to be determined.

While preferred embodiments have been set forth above, those skilled in the art who have reviewed the present disclosure will appreciate that other embodiments may be realized within the scope of the invention. For example, numerical values are illustrative rather than limiting, as are recitations of particular materials and of particular lens configurations. Also, the invention has applicability beyond the prostate and can be used for other imaging in the human or non-human animal body or for any other sort of photoacoustic imaging, including non-biological imaging. Therefore, the present invention should be construed as limited only by the appended claims.

1. A method for imaging an object internally in a subject, the method comprising:
inserting an imaging probe into a subject;
stimulating the object with laser light to produce ultrasound waves through the photoacoustic effect;
focusing the waves through an acoustic system comprising a multi-element acoustic lens within the probe, wherein the multi-element acoustic lens comprises a movable element or group of elements which provides the multi-element acoustic lens with variable focal length and magnification; and
imaging the focused waves in two dimensions.

2. (canceled)

3. The method of claim 1, wherein the focal length and magnification are varied in order to provide depth resolution.

4. The method of claim 1, wherein the multi-element acoustic lens is configured to correct aberrations so as to provide nearly diffraction-limited acoustic imaging.

5. The method of claim 1, wherein the multi-element acoustic lens comprises an element made of a hydrogel material.

6. The method of claim 1, wherein the object is a soft tissue.

7. The method of claim 6, wherein the soft tissue is in a prostate.

8. The method of claim 1, wherein the acoustic system further comprises an acoustic mirror.

9. The method of claim 8, wherein the acoustic mirror is curved.

10. An insertable probe for imaging an object within a subject, the insertable probe comprising:
a housing;
an acoustic and optical window in the housing;
optics for applying laser light to the object to produce ultrasound waves through the photoacoustic effect;
an acoustic system for focusing the waves, the acoustic system comprising a multi- element acoustic lens within the insertable probe, wherein the multi-element acoustic lens comprises a movable element or group of elements which provides the multi-element acoustic lens with variable focal length and magnification; and
a detector array, disposed so that the acoustic system focuses the waves onto the detector array, for imaging the focused waves in two dimensions.

11. (canceled)

12. The insertable probe of claim 10, wherein the multi-element acoustic lens is configured to correct aberrations so as to provide nearly diffraction-limited acoustic imaging.

13. The insertable probe of claim 10, wherein the multi-element acoustic lens comprises an element made of a hydrogel material.

14. The insertable probe of claim 10, wherein the acoustic system further comprises an acoustic mirror.

15. The insertable probe of claim 14, wherein the acoustic mirror is curved.

16. A multi-element acoustic lens positioned within an insertable probe, comprising:
a plurality of acoustic lens elements positioned within a probe suitable for insertion into a subject, wherein at least one of the plurality of acoustic lens elements is configured as a movable element or group of elements which provides the multi-element acoustic lens with variable focal length and magnification, the plurality of acoustic lens elements comprising:
at least one acoustic lens element having a positive power; and
at least one acoustic lens element having a negative power;
the plurality of lens elements being arranged to be coaxial.

17. (canceled)

18. The multi-element acoustic lens of claim 16, wherein the multi-element acoustic lens is configured to correct aberrations so as to provide nearly diffraction-limited acoustic imaging.

19. The multi-element acoustic lens of claim 16, wherein the multi-element acoustic lens comprises an element made of a hydrogel material.

20. The method of claim 1, further comprising using an actuator to move the movable element or group of elements to vary the focal length and magnification.

21. The insertable probe of claim 10, further comprising an actuator for moving the movable element or group of elements to vary the focal length and magnification.

22. The multi-element acoustic lens of claim 16, further comprising an actuator for moving the movable element or group of elements to vary the focal length and magnification.

23. The method of claim 1, wherein the subject is a human.
